(19) United States
(12) Patent Application Publication
(10) Pub. No.: US 2011/0244062 A1
(43) Pub. Date: Oct. 6, 2011

(54) MULTIPLICATION OF THE EFFICACY OF ANTI-INFECTIOUS AGENTS BY A COMPOSITION COMPRISING AN EXTRACT MADE FROM DEHYDRATED AND PARTIALLY SODIUM-FREE SEAWATER

(75) Inventors: Pierre LAGNY, Kildare (IE); Jean-Jacques HERAULT, London (GB)

(73) Assignee: AROMAPHARM IRELAND LTD., Dublin (IE)

(21) Appl. No.: 13/026,347

(22) Filed: Feb. 14, 2011

(30) Foreign Application Priority Data
Feb. 23, 2010 (EP) 10 305 180.1
Dec. 17, 2010 (EP) 10 195 669.6

(51) Int. Cl. A01N 65/36; A01P 1/00; A01P 3/00; A01N 43/90; A61K 31/14; A61K 31/43; A61K 36/752; A61P 31/12; A61P 31/10; A61P 31/04; A61K 31/545; A61K 31/65; A01N 37/18; A61K 31/4704; A01N 43/42; A61K 31/155; A01N 47/44; A61K 31/11; A01N 35/02

(52) U.S. Cl. 424/736; 514/643; 514/192; 514/200; 514/152; 514/312; 514/635; 514/703

(57) ABSTRACT

The present invention relates to a composition inhibiting or destroying at least one living or unicellular organism, which comprises at least one anti-infectious agent and one activating agent, which consists of an extract made from dehydrated and partially sodium-free seawater or an extract made from a dried fossil sea with a similar composition. The present invention also relates to the utilization of such a composition.

### FIELD

The present invention relates in general to the destruction and/or the inhibition of living or unicellular organisms such as protozoa, microbes, bacteria, gametes, fungi, yeasts, parasites or others.

### BACKGROUND

Many substances inhibiting or destroying unicellular living organisms are already known, and these include the surfactant agents such as quaternary ammonium salts. In particular, it is known that quaternary ammonium halides such as benzalkonium chloride (or alkyl-benzyl-dimethyl-ammonium chloride), alone or in combination with other active ingredients, are advantageous in these applications (see, for example, British Patents GB1 554 615, French Patents FR 2 431 859, FR 2 483 177, FR 2 379 508, FR 2 384 497, FR 2 457 641, FR 2 573 624, FR 2 418 221, FR 2 562 888, European Patents EP 0 243 713, EP 0 175 338, EP 0 132 963, EP 0 127 131, EP 0 094 562, EP 0 076 136, EP 0 068 399, EP 0 037 593, and international applications WO 84/00877 and WO 84/02649).

Moreover, the prior art has already provided many processes for manufacturing these quaternary ammonium salts (among quaternary ammonium halides), directly applicable to the chlorides and/or iodides, and/or bromides, and/or chloro-iodites (see above patents and also French Patents FR 2 472 558, FR 2 033 044 and European Patents EP 0 094 552 and EP 0 012 296).

Ammonium fluoride and processes for preparing and purifying it are also known (see, for example, French Patents FR 2 244 713, 2 253 710 and European Patent EP 0 002 016), as are perfluorinated or polyfluorinated quaternary ammonium salts (for example, French Patents 2 038 421, 2 051 095, 2 153 489 and European Patents EP 0 073 760, EP 0 100 478, EP 0 100 477, and EP 0149 172).

In European Patent EP 0308564, the problem of providing a composition that completely inhibits or destroys unicellular living organisms and which is simultaneously applicable to living organisms, human beings, animals or plants, without thereby causing harmful side effects, is raised. The solution to this problem described in EP 0308564 consists in a composition containing at least a myristalkonium fluoride, which may be used in human beings or animals. However, this composition has the disadvantage of causing an irritation at the site of injection, except when it is used through central administration. This irritation, caused by the necessary amount of active ingredient in the composition, may be improved by the reduction of the amount of this active ingredient when it is combined with an activating agent.

Moreover, European Patent EP 0310476 describes a pharmaceutical composition containing ionic and ionizable fluorine, and ionic or ionizable lithium, in particular lithium fluoride, i.e. a product of chemical origin.

### SUMMARY

The applicant has discovered that the addition of an extract made from dehydrated and partially sodium-free seawater or an extract made from a dried fossil sea with a similar composition, in a composition comprising an anti-infectious agent, allows in a surprising manner an increase in its inhibiting or destroying potency towards living or unicellular organisms.

On the other hand, such a compound only has by itself a minor potency to inhibit or destroy an infection and it only has a catalytic effect towards the anti-infectious agents used (also called active ingredients in the present application).

Thus, more particularly, the present invention relates to a composition that inhibits or destroys at least one living or unicellular organism, and which comprises at least one anti-infectious agent, and one activating agent consisting of an extract made from dehydrated and partially sodium-free seawater or an extract made from a dried fossil sea with a similar composition.

### DETAILED DESCRIPTION

Activating Agent

The activating agent of the composition according to the invention, as it is an extract made from the dehydration and partial withdrawal of sodium chloride from seawater, essentially contains the 83 known elements which are present in seawater, or those which are present in any other extract made from a dried fossil sea with a similar composition (example: Himalaya salt).

The activating agent is preferably present in the composition according to the invention in the amount of 0.01 ppm (or mg/L) to 120 ppm (or mg/L) to obtain the surprising effects described and claimed in the present invention (i.e. a noticeable activation of the inhibiting potency of the active ingredient (or ingredients) present in the composition according to the invention).

Anti-Infectious Agent

The composition according to the invention may be normally applied to all the existing anti-infectious active ingredients (or agents) whatever their origin, vegetable or animal or of chemical synthesis, and in particular to the following anti-infectious agents: all the bactericidal, antibiotic, fungicidal, virucidal, antiparasitic compounds, surface disinfectants, phyto-sanitary compounds, whatever their chemical, vegetable or animal origin.

As antibiotics usable within the scope of the present invention, penicillins, cephalosporins, cyclines, aminosides, macrolides, sulfamides, quinolones, phenicolated antibiotics, etc. may be more especially mentioned.

As antibiotics usable within the scope of the present invention, lincosamides, synergistines, glycopeptidic antibiotics, fusidic acid, fosfomycin, rifampicin, etc. may also be mentioned.

As virucides usable within the scope of the present invention:
the chemical virucides, which may be chosen among quaternary ammonium compounds, for example benzalkonium chloride, and tetradecyl-dimethyl-benzyl-ammonium fluoride (or myristalkonium fluoride), etc. and
the virucides of vegetable origin, which may be chosen among the essential oils of oregano (Origanum majorana), thyme (Thymus vulgaris), and savory (Satureja montana), etc., or among plant extracts such as grapefruit (Citrus paradisi) seed extract, or
the virucides of animal origin such as Propolis,
   may be mentioned in particular.

As bactericides usable within the scope of the present invention, the chemical bactericides, such as quaternary ammonium compounds, biguanides, carbanilides, phenolic compounds, chlorinated compounds, and glutaraldehyde, etc. may be mentioned in particular.

According to a particularly avantageous mode of realization of the present invention, the anti-infectious agent is the following fluorinated quaternary ammonium compound: tetradecyl-dimethyl-benzyl-ammonium fluoride (or TDBAF or myristalkonium fluoride). With such an anti-infectious agent present in the composition according to the invention, this one may be administered by injection.

TDBAF belongs to the family of the cationic quaternary ammonium compounds which is known for its potent antibacterial properties, and benzalkonium chloride or alkyl-dimethyl-benzyl-ammonium chloride is one of its more active compounds. The activity of TDBAF was described in the following publication: “Efficacy of a new quaternary ammonium compound against TB”, of Byrne C., Healy T M, in Jr J Med Sci, 1999 January-March; 168 (1): 45-6 (PMID: 10098344) [PubMed—indexed for Medline]. [TB=Mycobacterium tuberculosis].

As fungicides usable within the scope of the present invention:
the chemical fungicides, for example econazole, ketoconazole, ciclopirox olamine, etc., and
the fungicides of vegetable origin, for example the essential oils of rose geranium (Pelargonium asperum), palmarosa (Cymbopogon martinii variety motia), and spike lavender (Lavendula latifolia), etc., or plant extracts such as grapefruit (Citrus paradisi) seed extract, or
the fungicides of animal origin such as Propolis,
   may be mentioned in particular.

As phyto-sanitary product usable within the scope of the present invention, Neem oil (extracted from Azadirachta indica) may be mentioned in particular.

The anti-infectious agent according to the invention may be of chemical origin (as it is the case for the antibiotics, the fungicides and the virucides), or of vegetable or animal origin.

In the present invention, product of chemical origin means all the products prepared by total or partial synthesis.

In the present invention, product of vegetable origin means the whole product of the plant or any type of plant extract, whatever the method of extraction or of production.

In the present invention, product of animal origin means a product the origin of which is neither chemical, nor vegetable, but animal, such as Propolis which is a product made by bees.

As products of vegetable origin usable within the scope of the present invention as anti-infectious agent, grapefruit seed extract, essential oils, macerated extracts, tinctures, dry extracts, aqueous extracts, and total extracts of plants, and any other type of plant extract, whatever the method of extraction or of production, are more particularly mentioned.

As product of animal origin usable within the scope of the present invention as anti-infectious agent, Propolis, which is a complex made by bees with their secretions and a series of resinous, gummy and balsamic substances is more particularly mentioned.

The composition according to the invention may be used in the fields of surface disinfection, hygiene products or cosmetic products, insofar as they contain an anti-infectious agent, as well as of phyto-sanitary products and food preservative agents.

Hence, the composition according to the invention may contain in addition one or several ingredients (or additional compounds) which are classically used in the concerned fields. The amounts of these various additional ingredients are those usually used in the concerned fields.

Of course, the specialist in the art will make sure to choose the possible compound or compounds to add to the composition according to the invention (in particular according to the envisaged utilization or application), as well as their concentration, so that the avantageous properties intrinsically linked should not be, or should not be substantially altered by the envisaged addition. In particular, the avantageous properties of the anti-infectious agent will not have to be damaged by this (these) additional compound(s).

Thus, the object of the present invention is also to use the composition according to the invention in a cosmetic or body hygiene product.

The composition according to the invention may also be used in a phyto-sanitary product.

More generally, the composition according to the invention may be applied to the human or animal body as a whole, or to vegetable organisms, or to inert surfaces to disinfect.

The present invention also relates to a composition which comprises at least one anti-infectious agent and one activating agent consisting of an extract made from dehydrated and partially sodium-free seawater or an extract made from a dried fossil sea with a similar composition, for its utilization as a medicinal product in the infectious diseases. The addition of the activating agent to said composition results in an increase in the inhibiting or destroying potency of the anti-infectious agent. This activating agent is preferably present in said composition in the amount of 0.01 mg/L to 120 mg/L.

Such a composition according to the invention allows the activation of the activity of the anti-infectious agent, which in particular may be an antibiotic, bactericidal, fungicidal, or virucidal activity, which may relate to the whole human or animal body. The importance of the activation may vary according to the microorganisms encountered but not the reality of the effect.

More particularly, such a composition according to the invention allows the activation of a bactericidal activity to combat an infectious agent of the bacterial families. This effect may be applied to all bacterial families: gram-positive and gram-negative cocci, gram-positive and gram-negative bacilli, acid- and alcohol-fast bacilli, spiral bacteria, etc. This utilization for bactericidal purposes allows not only the inhibition of bacterial resistances (for example, in general, beta-lactamase-positive bacteria become beta-lactamase-negative) in increasing the efficacy of the active ingredients, but also allows the reduction of the doses of active ingredients, whatever the active ingredient, and thus it minimizes their possible adverse side effects in human beings, animals, plants and in the environment as a whole.

More particularly, such a composition according to the invention allows the activation of a fungicidal activity, which may be applied to the different forms of fungi: dermatophytes, yeasts, etc.

More particularly, such a composition according to the invention allows the activation of virucidal active ingredients such as tetradecyl-dimethyl-benzyl-ammonium fluoride mentioned above.

A preferred composition according to the invention consists in the addition of an extract made from dehydrated and partially sodium-free seawater (food compound) to an anti-infectious agent of vegetable origin such as grapefruit seed extract, in order to increase the inhibiting potency of this one. Due to its nature, this composition, which contains food compounds, represents an alternative of complete harmlessness to the usually used anti-infectious agents.

The invention is illustrated in greater detail in the following examples. In the examples, except where otherwise stated, all the amounts are expressed as a percentage in volume, or as mg/L, or as μg or as μL of composition.

### EXAMPLES

### Products

Microorganisms:
Staphylococcus aureus ATCC 29213,
MRSA (meticillin resistant Staphylococcus aureus) ATCC 1026,
Streptococcus pneumoniae ATCC 49619,
Escherichia coli ATCC 25922,
Pseudomonas aeruginosa ATCC 27853,
Acinetobacter baumanii ATCC 19606,
Klebsiella pneumoniae ATCC 13883,
Serratia marcescens ATCC 43861,
Candida albicans, nosocomial strain,
Malassezia furfur, nosocomial strain.

Anti-Infectious Agent Tested:

Grapefruit seed extracts (Citrus paridisi), called hereafter GSE. Several extracts were used in the examples presented below in order to show the action of the activating agent, whatever the extract.

Activating Agent:

Extract made from dehydrated and partially sodium-free seawater, called hereafter PAM. It is an extract made from the dehydration and partial withdrawal of sodium chloride from seawater. It essentially contains the 83 known elements which are present in seawater. This activating agent was used in the above-mentioned examples but it may be replaced by any other extract made from a dried fossil sea with a similar composition (example: Himalaya salt).

### Material and Method

The aim is to determine the minimal inhibitory concentration (MIC) of an active ingredient (or anti-infectious agent) against eight bacterial strains and two fungal strains, each strain being inserted in an inoculum, in the presence of an agent activating the inhibiting potency, according to the present invention.

With regard to the present invention, the minimal inhibitory concentration (MIC) means the minimal concentration (in % vol/vol) of active ingredient necessary to inhibit 100 μL of inoculum.

To determine the MIC, the following compositions are prepared for each example:
a sample of positive control (called letter B in the test tables with bacteria and letter F in the test tables with fungi), which only contains the inoculum (100 μL) in the sterile culture medium of the microorganisms (100 μL).
a sample of negative control (called letter M in the test tables), which only contains the sterile culture medium of the microorganisms (200 μL), and
one or several inhibiting compositions according to the present invention, Ci (i corresponding to a whole number), containing:
   100 μL of a mixture of active ingredient and activating agent, or
   100 μL of a mixture of active ingredient and activating agent, with an excipient (dispersing agent),
to which 100 μL of inoculum are added.

The trials were carried out with the eight above-mentioned bacterial strains according to the method of microdilution in liquid medium described by the CLSI (Clinical and Laboratory Standards Institute), which was adapted if necessary. The method may be summarized as follows:
Distribute 100 μL of sterile culture medium in the pits of positive control of a 96-well, U-bottom plate.
Add to each appropriate pit of the 96-well plate:
1. 100 μL of an anti-infectious agent, or
95 μL of an anti-infectious agent and 5 μL of an activating agent, or
10 μL of a mixture of an anti-infectious agent and an activating agent in equal parts and 90 μL of an excipient (dispersing agent),
keeping to a total volume of 100 μL, except in the pits of positive control and negative control.
2. 100 μL of inoculum with an end concentration of 3−5×10⁵ CFU/mL (colony-forming units per milliliter), except in the pits of negative control.
Distribute 200 μL of sterile culture medium in the pits of negative control of the 96-well plate.
The plate is covered and incubated at 37° C. for 18 to 24 hours.
The results are read with a reading-minor or a spectrophotometer at the optical density of 620 nm.

The sensitivity threshold is not available in the CLSI (Clinical and Laboratory Standards Institute) guide.

The trials were also carried out with the two above-mentioned fungal strains according to the method of microdilution in liquid medium described by the CLSI (Clinical and Laboratory Standards Institute). The sensitivity threshold is not available in the CLSI guide.

These trials were carried out in the Laboratory of medical microbiology, Research and Development Unit of the University Hospital Center, Hotel Dieu Hospital, Montreal, Canada.

### Example 1

### Activation of the Inhibiting Activity of Grapefruit Seed Extract (Citrus paradisi) (GSE) on a Reference Bacterial Strain

In addition to the compositions of control described above (B positive control and M negative control), the following compositions were prepared in order to show the action of the activating agent:
a first comparative inhibiting composition CC₁, containing 100 μL of GSE,
a second comparative inhibiting composition C₁ according to the invention, containing 95 μL of GSE and 5 μL of PAM, i.e. a total volume of 100 μL,

The final concentration of PAM in the medium, i.e. in the 200 μL of each appropriate pit, is 60 mg/L. Therefore there are 12 μh of PAM in these 200 μL.
a third comparative inhibiting composition C₂ according to the invention, containing 95 μL of GSE and 5 μL of PAM, i.e. a total volume of 100 μL,

The final concentration of PAM in the medium, i.e. in the 200 μL of each appropriate pit, is 120 mg/L. Therefore there are 24 μg of PAM in these 200 μL.

100 μL of inoculum are added to each of the compositions described above.

For each of these compositions, the MIC of the active ingredient (GSE) was determined on a reference bacterial strain. The experimental results of these trials are presented in table 1 below.

TABLE 1

| | | | MIC (% v/v) of the bacterial strain by GSE alone | | |
|---|---|---|---|---|---|
| | | | or combined with PAM (60 or 120 mg/L)¹ | | |
| Reference | | | | C₁ | C₂ |
| bacterial | | | CC₁ | GSE | GSE |
| strain | Negative | Positive | GSE | (95 μL = 47.5%)² + | (95 μL = 47.5%)² + |
| (100 μL | control | control | (100 μL = | PAM (60 mg/L)¹ | PAM (120 mg/L)¹ |
| of inoculum) | M⁴ | B³ | 50%)² | (5 μL = 2.5%)² | (5 μL = 2.5%)² |
| Staphylococcus | − | + | 0.125 | 0.05 | 0.05 |
| aureus | | | | | |
| ATCC 29213 | | | | | |

¹Final concentrations of PAM in the medium, i.e. 12 μg or 24 μg of PAM in the 200 μL of each appropriate pit.
²Percentage of each product in the 200 μL of each appropriate pit.
³Positive control (inoculum): 100% bacterial growth.
⁴Negative control (sterile culture medium): 0% bacterial growth.

The experimental results presented in table 1 show that the addition of the activating agent PAM according to the invention allows an increase in the inhibiting potency of grapefruit seed extract (GSE), the MIC of which is divided by 2.5 (0.05 instead of 0.125) for the microorganism Staphylococcus aureus ATCC 29213.

In composition C₁ according to the invention, 12 μg of activating agent PAM are sufficient to increase the efficacy of 95 μL of grapefruit seed extract (GSE). Therefore, in composition C₁, an amount of PAM approximately 7 916 times lower than that of GSE is sufficient to increase the efficacy of this one.

In composition C₂ according to the invention, 24 μg of activating agent PAM are sufficient to increase the efficacy of 95 μL of grapefruit seed extract (GSE). Therefore, in composition C₂, an amount of PAM approximately 3 958 times lower than that of GSE is sufficient to increase the efficacy of this one.

In this example, the utilization of the activating agent PAM at a dose of 120 mg/L in composition C₂ did not yield a MIC of GSE lower than that observed with 60 mg/L in composition C₁: the MICs of C₁ and C₂ are 2.5 times lower than that of composition CC₁ (0.05 instead of 0.125). Therefore, the efficacy of the activating agent PAM according to the invention is not dose-dependent, but it is effective within precise limits.

These experimental results show that the inhibiting compositions according to the invention allow an increase in the inhibiting potency of the active ingredient used.

### Example 2

### Activation of the Inhibiting Activity of Grapefruit Seed Extract (Citrus paradisi) (GSE) on Seven Reference Bacterial Strains

In addition to the compositions of control described above (B positive control and M negative control), the following compositions were prepared in order to show the action of the activating agent:
a first comparative inhibiting composition CC₁, containing 100 μL of GSE,
a second comparative inhibiting composition C₁ according to the invention, containing 95 μL of GSE and 5 μL of PAM, i.e. a total volume of 100 μL,

The final concentration of PAM in the medium, i.e. in the 200 μL of each appropriate pit, is 60 mg/L. Therefore there are 12 μg of PAM in these 200 μL.

100 μL of inoculum are added to each of the compositions described above.

For each of these compositions, the MIC of the active ingredient (GSE) was determined on seven reference bacterial strains. The experimental results of these trials are presented in table 2 below.

TABLE 2

| | | | MIC (% v/v) of the bacterial | |
|---|---|---|---|---|
| | | | strains by GSE alone or | |
| | | | combined with PAM (60 mg/L)¹ | |
| Reference | | | | C₁ |
| bacterial | | | CC₁ | GSE |
| strains | Negative | Positive | GSE | (95 μL = 47.5%)² + |
| (100 μL | control | control | (100 μL = | PAM (60 mg/L)¹ |
| of inoculum) | M⁴ | B³ | 50%)² | (5 μL = 2.5%)² |
| MRSA | − | + | 0.125 | 0.029 |
| ATCC 1026 | | | | |
| Streptococcus | − | + | 0.125 | 0.029 |
| pneumoniae | | | | |
| ATCC 49619 | | | | |
| Escherichia | − | + | 0.125 | 0.05 |
| coli | | | | |
| ATCC 25922 | | | | |
| Pseudomonas | − | + | 0.125 | 0.05 |
| aeruginosa | | | | |
| ATCC 27853 | | | | |
| Acinetobacter | − | + | 0.125 | 0.05 |
| baumanii | | | | |
| ATCC 19606 | | | | |
| Klebsiella | − | + | 0.125 | 0.05 |
| pneumoniae | | | | |
| ATCC 13883 | | | | |
| Serratia | − | + | 0.125 | 0.05 |
| marcescens | | | | |
| ATCC 43861 | | | | |

¹Final concentration of PAM in the medium, i.e. 12 μg of PAM in the 200 μL of each appropriate pit.
²Percentage of each product in the 200 μL of each appropriate pit.
³Positive control (inoculum): 100% bacterial growth.
⁴Negative control (sterile culture medium): 0% bacterial growth.

In this trial on 7 reference bacterial strains (table 2), results similar to those observed in example 1 on Staphylococcus aureus ATCC 29213 are observed.

For the microorganisms MRSA (meticillin resistant Staphylococcus aureus) ATCC 1026 and Streptococcus pneumoniae ATCC 49619, in composition C₁ according to the invention, the addition of the activating agent PAM according to the invention, at a dose of 60 mg/L, allows an increase in the inhibiting potency of grapefruit seed extract (GSE), the MIC of which is divided by 4.3 (0.029 instead of 0.125). This example is of particular interest because the usual antibiotics are ineffective against this microorganism which is resistant, according to its name, and which causes an increasing anxiety in the scientific circles (see 20th European Congress on Clinical Microbiology and Infectious Diseases, Vienne, Apr. 10-13, 2010).

For the microorganisms Escherichia coli ATCC 25922, Pseudomonas aeruginosa ATCC 27853, Acinetobacter baumanii ATCC 19606, Klebsiella pneumoniae ATCC 13883 and Serratia marcescens ATCC 43861, in composition C₁ according to the invention, the addition of the activating agent PAM according to the invention, at a dose of 60 mg/L, allows an increase in the inhibiting potency of grapefruit seed extract (GSE), the MIC of which is divided by 2.5 (0.05 instead of 0.125).

In composition C₁ according to the invention, 12 μg of activating agent PAM are sufficient to increase the efficacy of 95 μL of grapefruit seed extract (GSE). Therefore, in this example, an amount of PAM approximately 7 916 times lower than that of GSE is sufficient to increase the efficacy of this one.

These experimental results show that the inhibiting composition according to the invention allows an increase in the inhibiting potency of the active ingredient used.

### Example 3

### Activation of the Inhibiting Activity of Grapefruit Seed Extract (Citrus paradisi) (GSE) on a Reference Bacterial Strain

In addition to the compositions of control described above (B positive control and M negative control), the following compositions were prepared in order to show the action of the activating agent:
a first comparative inhibiting composition CC₂, containing 100 μL of GSE,
a group of comparative inhibiting composition C₃, C₄, C₅, C₆, according to the invention, containing for a total volume of 100 μL:
   5 μL of GSE,
   5 μL of PAM, and
   90 μL of excipient (dispersing agent or Disper®).

The final concentration of PAM in the medium, i.e. in the 200 μL of each appropriate pit, is 4 mg/L for C₃, 6 mg/L for C₄, 8 mg/L for C₅ and 13 mg/L for C₆. Therefore the amount of PAM in these 200 μL is as follows: 0.8 μg for C₃, 1.2 μg for C₄, 1.6 μg for C₅ and 2.6 μg for C₆.

100 μL of inoculum are added to each of the compositions described above.

For each of these compositions, the MIC of the active ingredient (GSE) was determined on a reference bacterial strain. The experimental results of these trials are presented in table 3 below.

TABLE 3

| | | | MIC (% v/v) of the bacterial strain by GSE alone | | | | |
|---|---|---|---|---|---|---|---|
| | | | or combined with PAM (4 or 6 or 8 or 13 mg/L) ¹ | | | | |
| | | | and an excipient (dispersing agent or Disper ®) | | | | |
| | | | | C₃ | C₄ | C₅ | C₆ |
| | | | | GSE | GSE | GSE | GSE |
| Reference | | | | (5 μL = 2.5%) ² + | (5 μL = 2.5%) ² + | (5 μL = 2.5%) ² + | (5 μL = 2.5%) ² + |
| bacterial | | | | PAM (4 mg/L) ¹ | PAM (6 mg/L) ¹ | PAM (8 mg/L) ¹ | PAM (13 mg/L) ¹ |
| strain of | Negative | Positive | CC₂ | (5 μL = 2.5%) ² + | (5 μL = 2.5%) ² + | (5 μL = 2.5%) ² + | (5 μL = 2.5%) ² + |
| (100 μL | control | control | GSE | Disper ® | Disper ® | Disper ® | Disper ® |
| inoculum) | M ⁴ | B ³ | (100 μL = 50%) ² | (90 μL = 45%) ² | (90 μL = 45%) ² | (90 μL = 45%) ² | (90 μL = 45%) ² |
| Staphylococcus | − | + | 0.78 | 0.15 | 0.08 | 0.3 | 0.3 |
| aureus | | | | | | | |
| ATCC 29213 | | | | | | | |

¹ Final concentrations of PAM in the medium, i.e. 0.8 μg of PAM for C₃, 1.2 μg for C₄, 1.6 μg for C₅ and 2.6 μg for C₆, in the 200 μL of each appropriate pit.
² Percentage of each product in the 200 μL of each appropriate pit.
³ Positive control (inoculum): 100% bacterial growth.
⁴ Negative control (sterile culture medium): 0% bacterial growth.

The experimental results presented in table 3 show that the addition of the activating agent PAM according to the invention allows an increase in the inhibiting potency of grapefruit seed extract (GSE), even in the presence of an excipient of dispersant type (Disper®), on the microorganism Staphylococcus aureus ATCC 29213.

In composition C₃ according to the invention, the addition of the activating agent PAM, at a dose of 4 mg/L, allows an increase in the inhibiting potency of grapefruit seed extract (GSE), the MIC of which is divided by 5.2 (0.15 instead of 0.78).

In composition C₃ according to the invention, 0.8 μg of activating agent PAM are sufficient to increase the efficacy of 5 μL of GSE. Therefore, in this composition, an amount of PAM approximately 6 250 times lower than that of GSE is sufficient to increase the efficacy of this one.

In composition C₄ according to the invention, the addition of the activating agent PAM, at a dose of 6 mg/L, allows an increase in the inhibiting potency of grapefruit seed extract (GSE), the MIC of which is divided by 9.75 (0.08 instead of 0.78).

In composition C₄ according to the invention, 1.2 μg of activating agent PAM are sufficient to increase the efficacy of 5 μL of GSE. Therefore, in this composition, an amount of PAM approximately 4 166 times lower than that of GSE is sufficient to increase the efficacy of this one.

In compositions C₅ and C₆ according to the invention, the addition of the activating agent PAM, at doses of 8 mg/L and 13 mg/L, allows an increase in the inhibiting potency of grapefruit seed extract (GSE), the MIC of which is divided by 2.6 (0.3 instead of 0.78).

In composition C₅ according to the invention, 1.6 μg of activating agent PAM are sufficient to increase the efficacy of 5 μL of GSE. Therefore, in this composition, an amount of PAM approximately 3 125 times lower than that of GSE is sufficient to increase the efficacy of this one.

In composition C₆ according to the invention, 2.6 μg of activating agent PAM are sufficient to increase the efficacy of 5 μL of GSE. Therefore, in this composition, an amount of PAM approximately 1 923 times lower than that of GSE is sufficient to increase the efficacy of this one.

In composition C₅, the activating effect is observed with 8 mg/L of PAM. In composition C₆, the increase in the concentration of PAM from 8 mg/L to 13 mg/L (i.e. an increase by more than 50%) does not yield an increase in the inhibiting potency which is 0.3 for both compositions C₅ and C₆: the MICs of C₅ and C₆ are 2.6 times lower than that of composition CC₂ (0.3 instead of 0.78).

In this example, although an amount of EPP lower than that in examples 1 and 2 (5 μL instead of 95 μL) and an excipient of dispersing type are used, the activating agent PAM according to the invention allows an increase in the efficacy of GSE.

These experimental results show that the inhibiting compositions according to the invention allow an increase in the inhibiting potency of the active ingredient used.

### Example 4

### Activation of the Inhibiting Activity of Grapefruit Seed Extract (Citrus paradisi) (GSE) on Two Nosocomial Fungal Strains

In addition to the compositions of control described above (F positive control and M negative control), the following compositions were prepared in order to show the action of the activating agent:
a first comparative inhibiting composition CC₂, containing 100 μL of GSE,
a second comparative inhibiting composition C₁, according to the invention, containing 95 μL of GSE and 5 μL of PAM, i.e. a total volume of 100 μL.

The final concentration of PAM in the medium, i.e. in the 200 μL of each appropriate pit, is 60 mg/L. Therefore there are 12 μg of PAM in these 200 μL.

100 μL of inoculum are added to each of the compositions described above.

For each of these compositions, the MIC of the active ingredient (GSE) was determined on two nosocomial fungal strains. The experimental results of these trials are presented in table 4 below.

TABLE 4

| | | | MIC (% v/v) of the fungal | |
|---|---|---|---|---|
| | | | strains by GSE alone | |
| Nosocomial | | | or combined with PAM (60 mg/L)¹ | |
| fungal | | | CC₂ | C₁ |
| strains | Negative | Positive | GSE | GSE (95 μL = 47.5%)² + |
| (100 μL | control | control | (100 μL = | PAM (60 mg/L)¹ |
| of inoculum) | M⁴ | F³ | 50%)² | (5 μL = 2.5%)² |
| Candida | − | + | 0.25 | 0.06 |
| albicans | | | | |
| Malassezia | − | + | 0.50 | 0.05 |
| furfur | | | | |

¹Final concentration of PAM in the medium, i.e. 12 μg of PAM in the 200 μL of each appropriate pit.
²Percentage of each product in the 200 μL of each appropriate pit.
³Positive control (inoculum): 100% fungal growth.
⁴Negative control (sterile culture medium): 0% fungal growth.

The experimental results presented in table 4 show that the addition of the activating agent PAM according to the invention allows an increase in the inhibiting potency of grapefruit seed extract (GSE), on two nosocomial fungal strains of Candida albicans and Malassezia furfur.

For the fungi Candida albicans, in composition C₁ according to the invention, the addition of the activating agent PAM, at a dose of 60 mg/L, allows an increase in the inhibiting potency of grapefruit seed extract (GSE), the MIC of which is divided by 4.16 (0.06 instead of 0.25).

For the fungi Malassezia furfur, in composition C₁ according to the invention, the addition of the activating agent PAM, at a dose of 60 mg/L, allows an increase in the inhibiting potency of grapefruit seed extract (GSE), the MIC of which is divided by 10 (0.05 instead of 0.50).

These experimental results show that the inhibiting composition according to the invention allows an increase in the inhibiting potency of the active ingredient used not only on bacteria, as demonstrated in the examples above, but also on fungi.

1. Composition inhibiting or destroying at least one living or unicellular organism, which comprises at least one anti-infectious agent and one activating agent, which is an extract made from dehydrated and partially sodium-free seawater or an extract made from a dried fossil sea with a similar composition.

2. Composition according claim 1, characterized in that the activating agent is present in said composition in the amount of 0.01 mg/L to 120 mg/L.

3. Composition according claim 1 or 2, characterized in that the anti-infectious agent is chosen among antibiotics, bactericides, fungicides, virucides, antiparasitics, surface disinfectants, and phyto-sanitary compounds, whatever their chemical, vegetable or animal origin.

4. Composition according to claim 3, characterized in that the anti-infectious agent is an antibiotic chosen among penicillins, cephalosporins, cyclines, aminosides, macrolides, sulfamides, quinolones, and phenicolated antibiotics.

5. Composition according to claim 3, characterized in that the anti-infectious agent is a chemical virucide chosen among quaternary ammonium compounds, or a virucide of vegetable origin chosen among the essential oils of oregano, thyme, savory or among plant extracts, or a virucide of animal origin.

6. Composition according to claim 3, characterized in that the anti-infectious agent is a chemical bactericide chosen among quaternary ammonium compounds, biguanides, carbanilides, phenolic compounds, chlorinated compounds, and glutaraldehyde.

7. Composition according to claim 5 or 6, characterized in that the anti-infectious agent is tetradecyl-dimethyl-benzyl-ammonium fluoride, and said composition may be administered by injection.

8. Composition according to claim 3, characterized in that the anti-infectious agent is a chemical fungicide chosen among econazole, ketoconazole, ciclopirox olamine, or a fungicide of vegetable origin chosen among the essential oils, for example the essential oils of rose geranium (Pelargonium asperum), palmarosa (Cymbopogon martinii variety motia), and spike lavender (Lavendula latifolia), or among plant extracts, or a fungicide of animal origin.

9. Composition according to claim 3, characterized in that the anti-infectious agent is a a phyto-sanitary product.

10. Composition according to claim 3, characterized in that the anti-infectious agent is a product of vegetable origin chosen among essential oils, macerated extracts, tinctures, dry extracts, aqueous extracts, total extracts of plants, and any type of plant extract, whatever its method of extraction or of production, or a product of animal origin.

11. Utilization of the composition as it is defined according to any single claim from 1 to 10, in a cosmetic or hygiene product, insofar as it contains an anti-infectious agent, or in a phyto-sanitary product, or food preservative agents.

12. Composition comprising at least one anti-infectious agent and one activating agent, which consits of an extract made from dehydrated and partially sodium-free seawater or an extract made from a dried fossil sea with a similar composition, for its utilization as a medicinal product in the infectious pathologies.

13. Composition according to claim 12, characterized in that the activating agent is present in said composition in the amount of 0.01 mg/L to 120 mg/L.

14. Composition according to claim 12 or 13, for its utilization as a medicinal product to combat infectious diseases of viral, or bacterial or fungal origin, concerning the whole human or animal body.
